(19) United States
(12) Patent Application Publication
(10) Pub. No.: US 2026/0334971 A1
(43) Pub. Date: Oct. 29, 2026

(54) THROMBUS REMOVAL SYSTEMS AND ASSOCIATED METHODS

(71) Applicant: Shifamed Holdings, LLC, Campbell, CA (US)

(72) Inventors: Aadel AL-JADDA, San Carlos, CA (US); Amr SALAHIEH, Saratoga, CA (US); Paul GUNNING, Montara, CA (US); Muralidharan SRIVATHSA, Santa Clara, CA (US); Uday ILLINDALA, Los Gatos, CA (US)

(21) Appl. No.: 19/106,171

(22) PCT Filed: Aug. 24, 2023
(86) PCT No.: PCT/US2023/072796

§ 371 Date: Feb. 24, 2025

Related U.S. Application Data
(60) Provisional application No. 63/373,420, filed on Aug. 24, 2022.
(60) Provisional application No. 63/380,851, filed on Oct. 25, 2022.

(51) Int. Cl. A61B 17/221; A61B 17/00; A61B 17/22; A61B 90/00

(52) U.S. Cl. CPC A61B 17/221; A61B 90/39; A61B 2017/00867; A61B 2017/22079; A61B 2017/2215; A61B 2090/3966

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application No. 63/373,420, titled “THROMBUS REMOVAL SYSTEMS AND ASSOCIATED METHODS”, filed on Aug. 24, 2022, and U.S. provisional patent application No. 63/380,851, titled “THROMBUS REMOVAL SYSTEMS AND ASSOCIATED METHODS”, filed on Oct. 25, 2022, which are both herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs.

Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure.

Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days.

Many thrombectomy devices have elements at the end (e.g. funnels) to assist with capturing clots, but can be difficult to visualize under imaging such as fluoroscopy. This makes it challenging for clinicians to guide the device to a target location and confirm positioning adjacent a clot to be removed.

More recently other tools like clot retrievers have been developed to treat DVT and pulmonary embolism (PE), but these tools are not being widely adopted because of their limited effectiveness and additional costs versus aspiration or the standard of case. Other recent developments focus on slicing or macerating the clot, but these mechanisms are designed to reduce the risk of the catheter clogging and do not address the problem of tough, large, organized clots.

There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4C illustrate various embodiments of a thrombus removal system including a saline source, an aspiration system, and one or more controls for controlling irrigation and/or aspiration of the system.

FIGS. 5A-5B illustrate one embodiment of a funnel of a thrombus removal system that includes imaging markers for visualization under real-time imaging.

FIGS. 6A-6B illustrate another embodiment of a funnel of a thrombus removal system that includes imaging markers for visualization under real-time imaging.

FIG. 7 illustrates yet another embodiment of a thrombus removal device funnel that includes imaging markers embedded in the funnel.

FIGS. 8A-8D illustrate additional embodiments of imaging markers that can be imbedded in a funnel to facilitate real-time monitoring or imaging of the imaging marker and/or funnel location.

FIGS. 9A-9D show imaging markers disposed within a funnel of a medical device.

FIGS. 10A-10D illustrate imaging markers that are asymmetrical in an axial direction to indicate an orientation of a medical device with respect to an imaging plane.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

A medical device is provided, comprising an elongate shaft comprising a distal end; and an expandable funnel disposed at or near the distal end, the funnel comprising a compliant material and at least one imaging marker disposed or encapsulated within the compliant material.

In one aspect, the device includes a funnel frame disposed within the compliant material.

In some aspects, the at least one imaging marker is supported only by the compliant material.

In one aspect, the at least one imaging marker is supported only by the compliant material and not by the funnel frame.

In other aspects, the at least one imaging marker comprises a radiopaque material.

In some aspects, the at least one imaging marker comprises platinum iridium.

In one aspect, the at least one imaging marker comprises a coil, wherein the coil is wound around at least a portion of the funnel frame.

In some aspects, the funnel frame comprises a shape memory material.

In one aspect, the funnel frame includes a distal end arranged in a chevron pattern.

In some aspects, the at least one imaging marker is disposed around at least one segment of the chevron pattern.

In one aspect, the at least one imaging marker comprises a circumferential coil disposed near a distal end of the funnel.

In some aspects, the at least one imaging marker comprises a plurality of imaging markers embedded in the compliant material at least partially distal to the funnel frame.

In another aspect, the at least one imaging marker is arranged generally along a longitudinal axis of the medical device.

In some aspects, the at least one imaging marker includes one or more axial flexibility features configured to improve or increase an axial flexibility of the at least one imaging marker.

In one aspect, the one or more axial flexibility features comprises a slit or cutout that is orthogonal to a longitudinal axis of the medical device.

In some aspects, the one or more imaging markers comprise a serpentine pattern.

In one aspect, the serpentine pattern includes gaps between the serpentine pattern than are generally orthogonal to a longitudinal axis of the one or more imaging markers.

In one aspect, the serpentine pattern does not include any free ends.

In some aspects, the one or more imaging markers are formed from a flat sheet of a Pt-Ir material.

A method for removing a thrombus from a blood vessel of a patient with a thrombus removal device is provided, the method comprising: introducing a distal portion of a thrombus removal device to a thrombus location in a blood vessel; expanding a funnel of the distal portion near the thrombus location; visualizing at least one imaging marker of the funnel; capturing the thrombus in the funnel; and removing at least a portion of the thrombus from the blood vessel.

In one aspect, the at least one imaging marker is supported only by a compliant material of the funnel.

In one aspect, the at least one imaging marker does not contact a frame structure of the funnel.

In some aspects, the method further comprises identifying an orientation feature of the at least one imaging marker to determine a position of the at least one imaging marker on the funnel.

In one aspect, visualizing the at least one imaging marker further comprises performing fluoroscopy imaging.

A method for visualizing a thrombus removal device is provided, the method comprising: introducing a distal portion of the thrombus removal device to a thrombus location in a blood vessel; expanding a funnel of the distal portion near the thrombus location; imaging at least one imaging marker of the funnel; and confirming a location of the at least one imaging marker near the thrombus location with the imaging.

In one aspect, visualizing the at least one imaging marker comprises performing fluoroscopy imaging of the at least one imaging marker.

In another aspect, the at least one imaging marker is radiopaque.

In some aspects, the at least one imaging marker is supported only by a compliant material of the funnel.

In one aspect, the at least one imaging marker does not contact a frame structure of the funnel.

In another aspect, the method includes identifying an orientation feature of the at least one imaging marker to determine a position of the at least one imaging marker on the funnel.

A thrombus removal device is provided, comprising: an elongate shaft comprising an aspiration lumen and a radiopaque distal end; an expandable element disposed at or near the radiopaque distal end and in fluid communication with the aspiration lumen; at least one radiopaque marker carried by the expandable element; wherein a radiopaque gap exists between the radiopaque distal end of the elongate shaft and the radiopaque marker carried by the expandable element.

In one aspect, the radiopaque gap comprises a portion of the device that is not clinically visible under fluoroscopy imaging.

A medical device adapted for insertion into a subject is provided, comprising: a frame or rigid structure; a compliant material supported by the frame or rigid structure; and at least one imaging marker disposed or encapsulated within the compliant material.

In some aspects, the at least one imaging marker is supported only by the compliant material.

In one aspect, the at least one imaging marker is supported only by the compliant material and not by the frame or rigid structure.

In another aspect, the at least one imaging marker comprises a radiopaque material.

In some aspects, the at least one imaging marker comprises platinum iridium.

In one aspect, the frame comprises a shape memory material.

In some aspects, the at least one imaging marker is arranged generally along a longitudinal axis of the medical device.

In one aspect, the at least one imaging marker includes one or more axial flexibility features configured to improve or increase an axial flexibility of the at least one imaging marker.

In some aspects, the one or more axial flexibility features comprises a slit or cutout that is orthogonal to a longitudinal axis of the medical device.

In another aspect, the one or more imaging markers comprise a serpentine pattern.

In some aspects, the serpentine pattern includes gaps between the serpentine pattern than are generally orthogonal to a longitudinal axis of the one or more imaging markers.

In one aspect, the serpentine pattern does not include any free ends.

In one aspect, the one or more imaging markers are formed from a flat sheet of a Pt-Ir material.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section.

Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance.

Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body.

The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

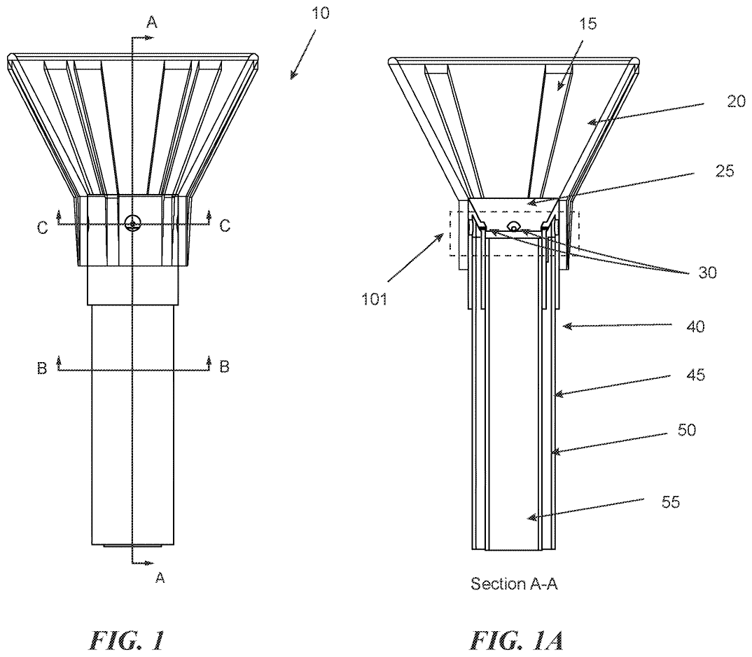

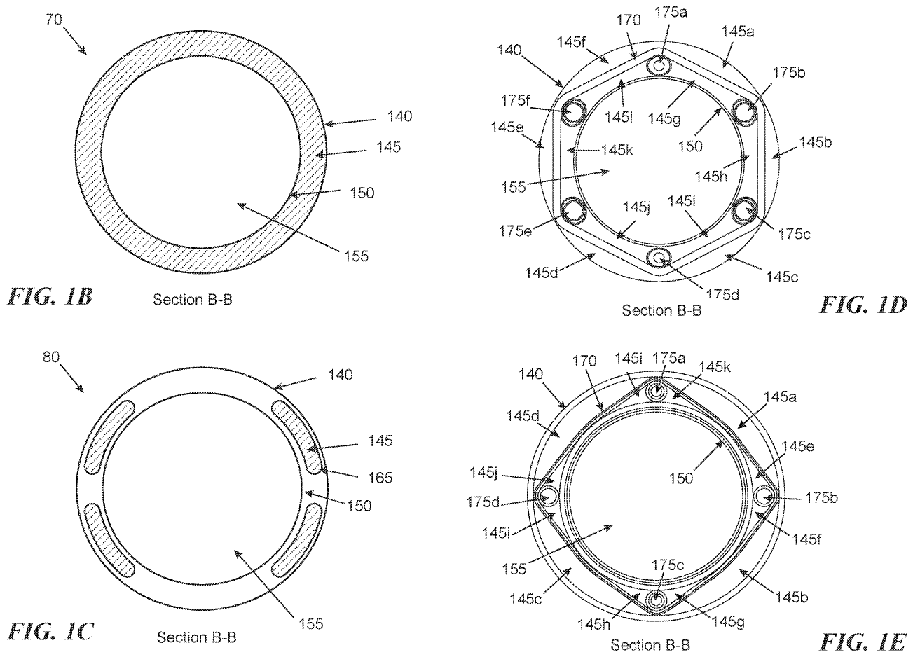

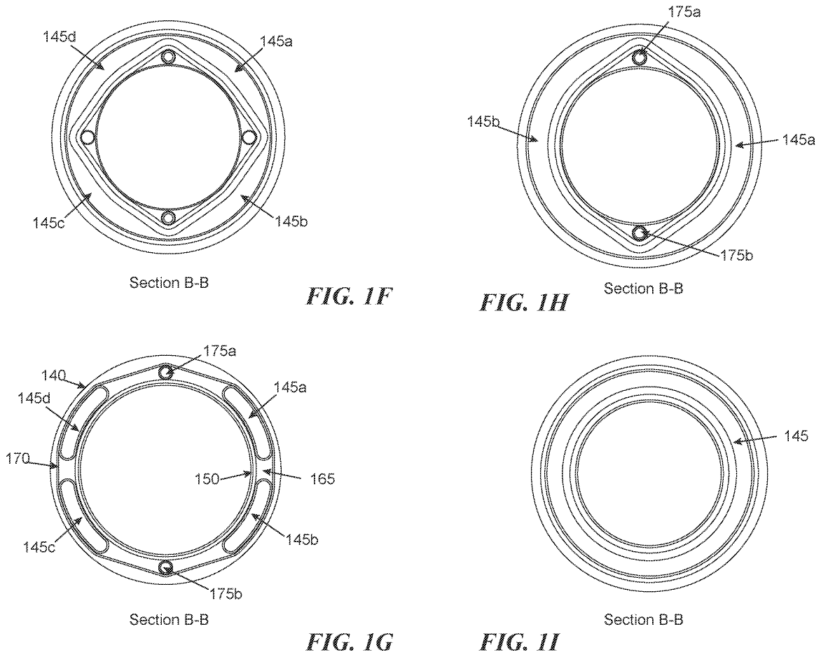

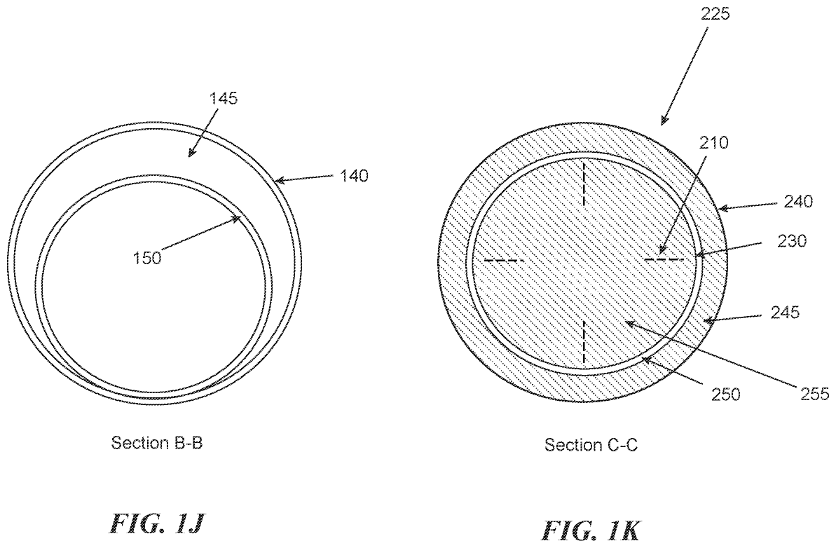

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus within a blood vessel and/or a tissue (e.g., vessel) wall to aid in thrombus fragmentation and/or removal. The funnel can have a variety of shapes and constructions as would be understood by one of skill from the description herein. The thrombus removal system may be delivered through a sheath to a thrombus site in a blood vessel with funnel 20 in a compressed configuration. Funnel 20 may self-expand as it is advanced out of the sheath and/or as the sheath is retracted from the funnel.

The example section A-A in FIG. 1A depicts a double walled thrombus removal device construction having a catheter 22 extending proximally from funnel 20 with an outer wall/tube 40 and an inner wall/tube 50. An aspiration lumen 55 is formed by the inner wall 50 and is centrally located. Aspiration lumen 55 communicates with a vacuum source, as described below. A generally annular volume forms at least one fluid lumen 45 between the outer wall 40 and the inner wall 50. The fluid lumen 45 is adapted for fluid communication with a fluid delivery mechanism, as described below. One or more apertures (e.g., nozzles, orifices, or ports) 30 are positioned in the thrombus removal system to be in fluid communication with the fluid lumen 45 and an irrigation manifold 25 at the base of funnel 20. In operation, the ports 30 are adapted to direct (e.g., pressurized) fluid toward thrombus material that is engaged with the distal portion 10 of the thrombus removal system to macerate, fragment, or cut the thrombus material. Aspiration lumen 55 pulls thrombus material along with fluid from ports 30 and blood from the blood vessel proximally to a receptacle outside of the patient, as described below.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-14l and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b.

Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150.

However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 at the base of the funnel that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

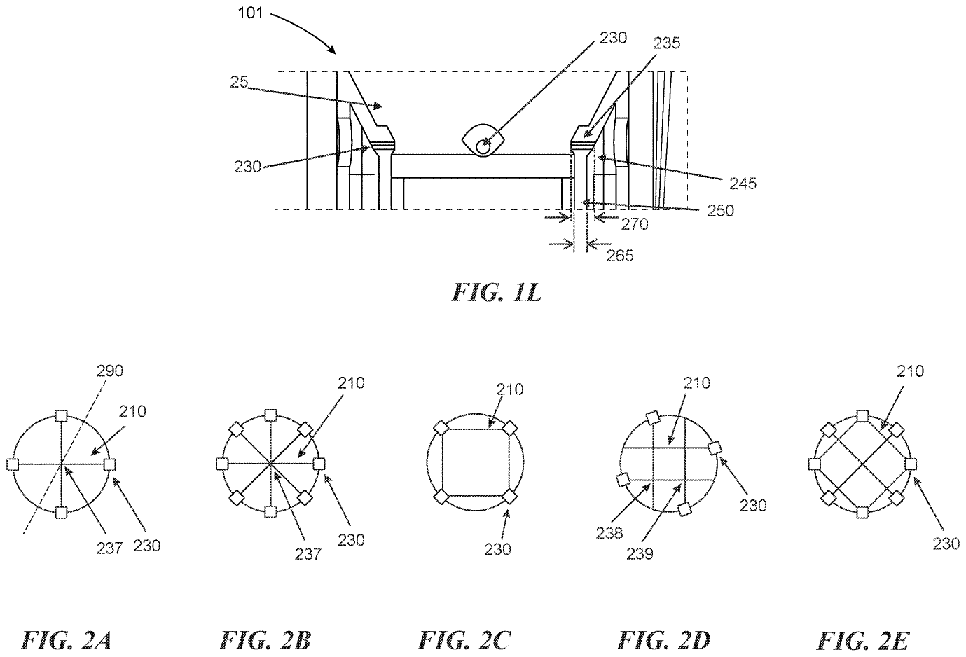

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams can be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

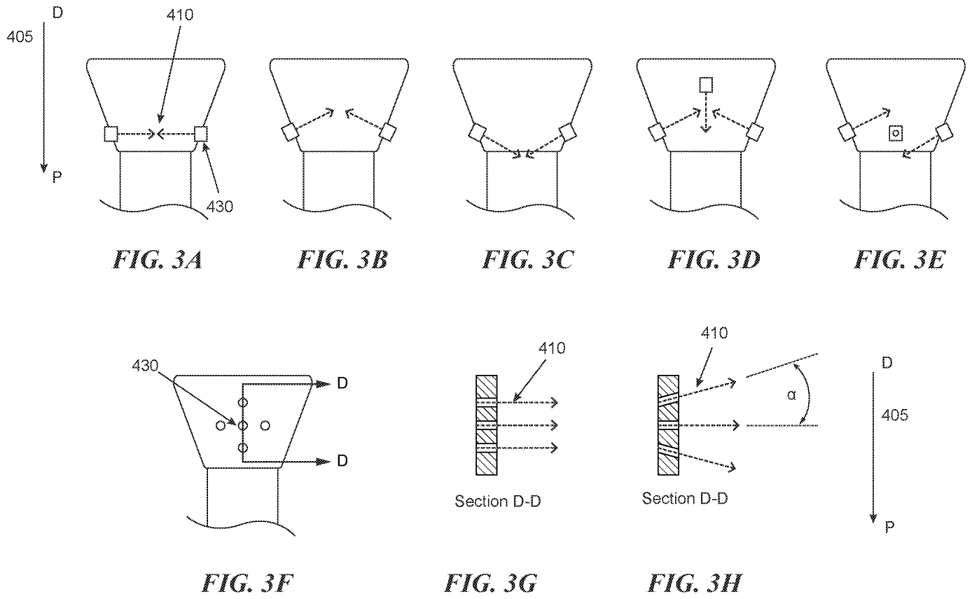

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

### Cavitation Generation

The exemplary system includes fluidic jets configured in a particular manner to enhance removal of clot. The exemplary fluid streams or jets have been shown in bench studies to dramatically improve removal of clot through various mechanisms of action optionally including, but not limited to, cavitation and water cutting. In contrast to conventional fluid mechanisms for thrombectomy, in some embodiments herein, fluid streams from respective ports 430 are delivered at sufficient flow rates (and patterns) to create cavitation and/or other preferential effects to improve removal of clot. In certain examples, the cavitation effect is created by large pressure drops and deceleration at the focal point and/or intersection point of at least two fluid streams. The cavitation may provide a source of turbulent kinetic energy that can be used to mechanically fractionate and/or liquefy thrombi or other target tissue structures. When the fluid velocity is sufficiently high, the material accumulates impact energy, which can cause deformation and fragmentation. This also may modify the surface properties of the clot to allow the material to be penetrated to enable cavitation within the clot. Collision or interaction of the high-speed jets creates hydrodynamic cavitation whereby a pressure drop below the vapor pressure of the liquid creates bubbles which eventually collapse with great mechanical energy in the cavitation field, causing a kind of implosion in the clot material. Further, with multiple jets directed towards a focal point or sufficiently near respective streams, the closing speed of the fluid particles is significantly higher (up to double) that of a single jet stream. This also forces fluid and/or particles out from the space between the fluid jets at high speed. The speed of the fluid jets is sufficiently high to create a pressure drop below the vapor pressure such that the fluid vaporizes. When pressure rises again the bubble collapses, which causes the cavitation. It has been found that the power of the exemplary system and cavitation effect significantly exceeds conventional fluid jet(s) and mechanical tools like rotating screws. In some examples, the collapse of the bubbles may generate heat in or around the target tissue, which may further promote breaking up of the clot. In bench studies systems in accordance with various embodiments were able to remove certain clot material that simple aspiration or water jetting were not. In other studies, the exemplary systems were able to remove clot material in a fraction of the time of conventional systems.

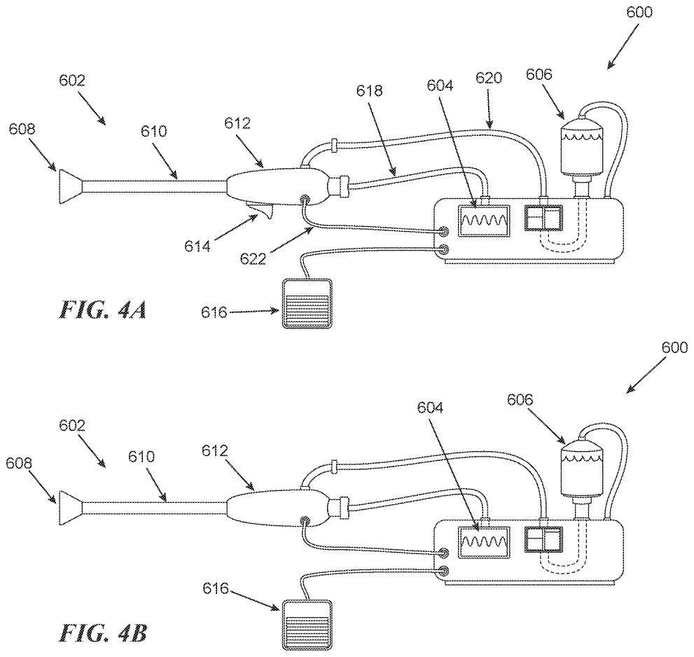

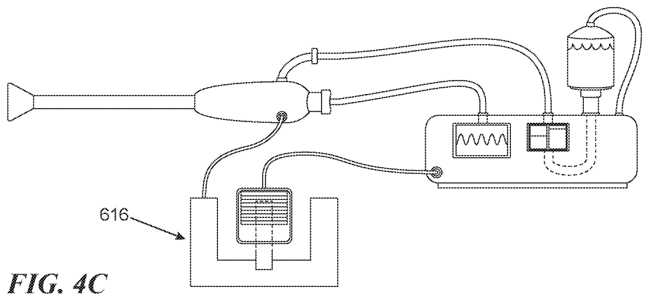

FIGS. 4A-4C illustrate various configurations of a thrombus removal system 600, including a thrombus removal device, 602, a vacuum source and cannister 604, a fluid source 606, and a pump 607. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 614, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source and cannister 604 can be coupled to the aspiration lumen of the device with a vacuum line 618. Any clots or other debris removed from a patient during therapy can be received by, and stored in the vacuum cannister 604. Similarly, the fluid source 606 (e.g., a saline bag) can be coupled to the fluid lumens of the device with a fluid line 620 for delivery of high-pressure fluid streams or jets at the base of funnel 608 to fragment thrombus material engaged by funnel 608, as described above.

During a thrombus removal procedure, pressurized fluid is delivered from fluid source 606 and pump 607 through fluid line 620 and fluid lumen 45 to manifold 25 and ports 30. Simultaneously, vacuum is applied to aspiration lumen 55 by vacuum source and cannister 604. At the beginning of the procedure, the material flowing proximally through aspiration lumen 55 to the vacuum source and cannister is primarily fluid delivered through ports 30 combined with any blood that is able to pass around the engaged thrombus. As the fluid jets begin to break up the thrombus, thrombus material is pulled proximally through aspiration lumen 55 along with the injected fluid and any blood that can pass into the funnel around the thrombus. After the thrombus has been broken up or macerated sufficiently to become dislodged from the blood vessel, the remaining thrombus material moves proximally toward the vacuum source and cannister 604.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

### Real-Time Imaging

The thrombus removal devices and systems described herein can include or incorporate features to facilitate real-time imaging and/or tracking of the device during delivery and therapy. In some implementations, the device can include features embedded or integrated into the working end, or more specifically, into the funnel of the device. These features can provide highly visible regions or markers under imaging guidance, such as under fluoroscopy or even ultrasound imaging.

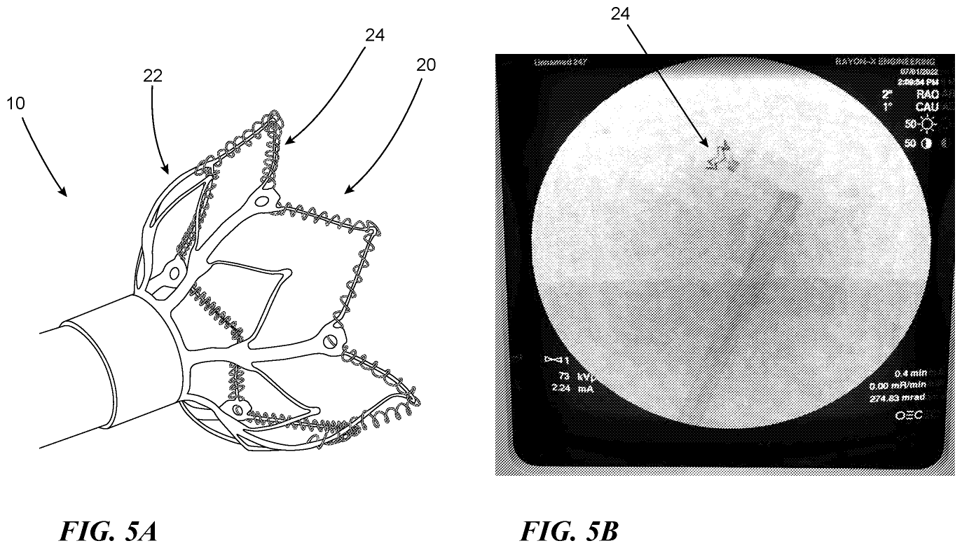

Referring to FIG. 5A, a thrombus removal device 10 is provided that can include a funnel 20 comprising a funnel frame 22. The funnel frame can provide, for example, a self-expanding structure configured to allow the funnel to transition from a collapsed delivery configuration to an expanded therapy configuration. In some embodiments, the funnel frame can comprise a shape memory material such as nitinol. Typically, the funnel frame is covered or embedded within a compliant material, such as a polycarbonate-based thermoplastic urethane (e.g., Chronoflex). The compliant material is not shown in this embodiment for ease of illustration and description. In some embodiments, the compliant material can comprise one or more layers. For example, a first layer of compliant material may be placed over an interior portion of the funnel frame and a second layer of the compliant material may be placed of an exterior portion of the funnel frame, to cover and protect the funnel frame from both the interior and exterior sides. The first or interior layer of the compliant material therefore makes up the interior surface of the funnel, and the second or exterior layer of the compliant material makes up the exterior surface of the funnel. While the compliant material is described as being formed in “layers”, it should be understood that any known technique for placing the compliant material on the funnel frame can be implemented, including spraying on the compliant material, or some combination of spraying, injection molding, or heat treating the compliant material to create the funnel.

Still referring to FIG. 5A, the thrombus removal device 10 can further include one or more imaging markers 24 in or within the funnel 20. In some embodiments, the imaging markers 24 can comprise a radiopaque material. In one specific embodiment, the imaging markers 24 can comprise a platinum-iridium (Pt—Ir) material. As shown, the imaging markers 24 comprise a plurality of coiled segments of Pt—Ir. In this embodiment, the coiled Pt—Ir segments are wound or coiled around the distal-most segments of the funnel frame 22. The illustrated funnel frame in this embodiment includes a “chevron” pattern on the distal-most portion of the frame, and each segment of the chevron pattern is then covered with the Pt—Ir coils. However, it should be understood that in other embodiments, all, some, or other portions of the funnel frame can be covered with the imaging markers 24.

FIG. 5B is a real-time image of the thrombus removal device of FIG. 5A. In this example, the image is a fluoroscopy image of the device and the imaging markers 24 can be clearly seen in the image, including the “chevron” pattern or shape of the markers at the distal end of the funnel.

FIGS. 6A-6B illustrate an alternative embodiment of imaging markers disposed on or in the funnel 20 of the thrombus removal device 10. In this embodiment, the imaging marker comprises a circumferential ring disposed at or near the distal end of the funnel. In contrast to the embodiment of FIG. 5A, where the imaging markers are wound around segments of the funnel frame itself, the imaging marker(s) of FIG. 6A instead are separate from the frame. In one example, the imaging markers are embedded in or supported by the compliant material 26 of the funnel 20. In some examples, the imaging markers are supported only by the compliant material of the funnel, and do not contact the funnel frame. The imaging marker itself can be similar to the marker of FIGS. 5A-5B and can comprise, for example, a length of Pt—Ir material. The marker can be a wound coil of Pt—Ir. While the embodiment of FIG. 6A shows a circumferential ring, in other embodiments it should be understood that the imaging markers can comprise a plurality of imaging marker segments that form a disjointed pattern around the perimeter of the funnel. The imaging markers can comprise, for example, lines, rectangles, dots, squares, or other shapes that are supported only by the compliant material of the funnel.

FIG. 6B shows a real-time image of the thrombus removal device of FIG. 6A. In this example, the image is a fluoroscopy image of the device and the circumferential imaging marker 24 can be clearly seen in the image at the distal end of the funnel.

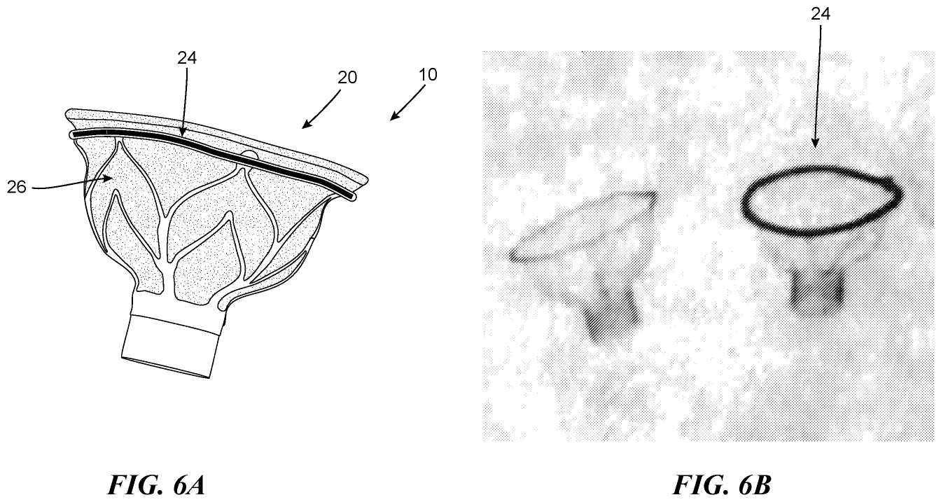

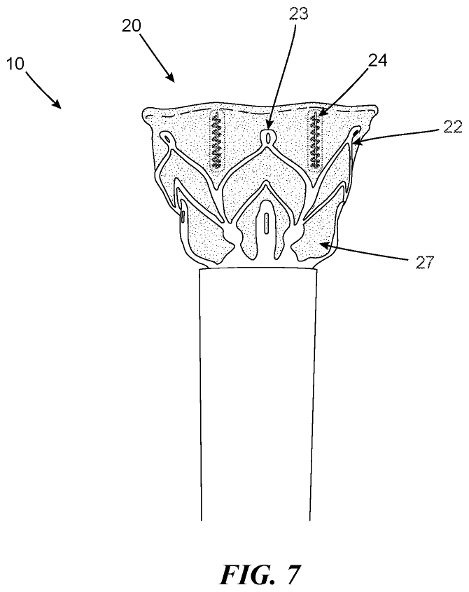

FIG. 7 is an additional embodiment of imaging markers 24 disposed on or in the funnel 20 of the thrombus removal device 10. In this embodiment, one or more imaging markers 24 are supported only by the compliant material of the funnel. In the illustrated embodiment, the one or more imaging markers do not contact nor are they supported by the funnel frame 22 of the funnel. As described above, in some embodiments the one or more imaging markers 24 may be embedded in the compliant material, which can be a polycarbonate-based thermoplastic urethane (e.g., Chronoflex). In some embodiments, the imaging markers can be supported between layers of the compliant material. Embedding or encapsulating the imaging markers in the compliant material reduces or eliminates the risk of the imaging markers from dislodging or breaking free from the funnel and escaping into a patient's vasculature.

The embodiment of FIG. 7 includes straight segments of coiled Pt-Ir material extending along a longitudinal axis of the thrombus removal device. In this embodiment, the imaging markers are placed in between the peaks 23 of a chevron pattern of the funnel frame 22. As shown, the imaging markers can extend distally beyond the peaks 23 of the funnel frame. In one embodiment, the funnel can include six imaging markers placed in this manner. However, it should be understood that any number of imaging markers can be used, including four, five, six, seven, eight, nine, ten, or more imaging markers. Other locations within the funnel are also contemplated. For example, one or more markers can be placed more proximally in the funnel, towards the aspiration lumen of the device, such as between the funnel frame 22 at location(s) 27.

It should be understood that the concept of supporting imaging markers within a compliant material such as Chronoflox is not limited to thrombus removal devices, but can be used in any catheter-based or implantable medical device. For example, catheters that do not include funnels can include imaging markers embedded within or supported only by a compliant material, such as a flexible/atraumatic distal tip, or a compliant coating placed over the catheter shaft. Other devices with rigid frames that may be covered with a compliant material can be used to support imaging markers in a similar manner, such as stents or valve frames. Placing the imaging markers away from a rigid frame provides a clear view of the imaging marker under fluoro and assists the user or medical provider in determining orientation and/or proper placement of the medical device.

Arranging the imaging markers in a longitudinal fashion as illustrated in FIG. 7 provides a visual indication to the user regarding the state of the funnel. For example, the user can see if the funnel is in a collapsed configuration or in an open or therapy configuration based on the orientation of the markers. For example, in a collapsed configuration, the imaging markers will generally be close to another and will typically be directed parallel to the longitudinal axis of the thrombus removal device. However, when the funnel is expanded, the distance between the imaging markers will increase. Additionally, some of the markers may not be parallel to the longitudinal axis of the thrombus removal device when the funnel is expanded, but instead will be oriented along with the shape of the expanded funnel.

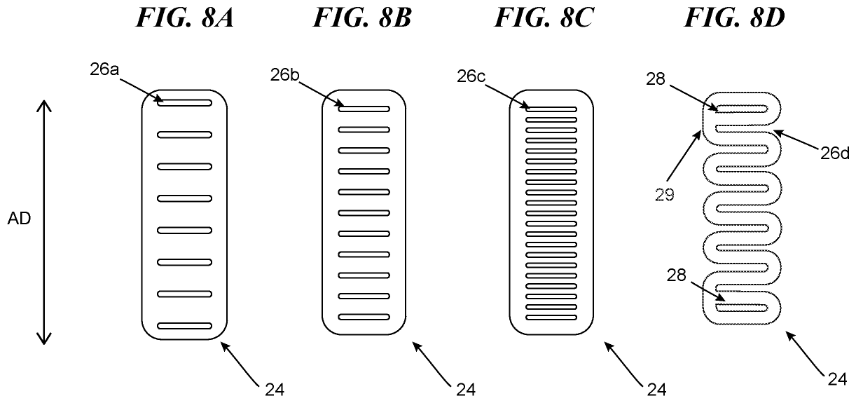

FIGS. 8A-8D illustrate additional embodiments of imaging markers 24 that can be configured to be disposed on or in the funnel 20 of the thrombus removal device described previously. As described above and illustrated in FIG. 7, any of the markers of FIGS. 8A-8D can be supported or disposed within a compliant material of the funnel, without contacting or being supported by a rigid or frame element of the funnel. In the illustrated embodiments, the imaging markers can be formed from flat sheets of Pt-Ir material. In one implementation, compliant material of the funnel can comprise a urethane material such as Chronoflex. In some examples, the structural components of the funnel, including the funnel frame and any imaging markers, can be laminated between two layers of the urethane material (e.g., Chronoflex). As described above, embodiments are presented herein where the imaging markers are supported only by the compliant material and not by any rigid structures of the device such as a frame (e.g., the funnel frame).

As described herein, the funnel can be configured to be collapsed or constrained during delivery, and can further be configured to be expanded during therapy. As such, the imaging markers of the present disclosure can include features configured to allow the imaging markers to be compliant or flexible in at least the axial or longitudinal direction when embedded in the compliant material of the funnel. FIGS. 8A-8D illustrate axial flexibility features in the imaging markers to allow for flexibility in the axial/longitudinal direction AD when embedded or mounted within the funnel. As shown, the cutouts or slits can be arranged to be orthogonal to the axial direction of the imaging markers (and funnel).

FIGS. 8A, 8B, and 8C illustrate imaging markers with axial flexibility features comprising slits or cutouts 26a, 26b, and 26c, respectively, to increase flexibility of the imaging markers in the axial direction. In these embodiments, the imaging markers comprise flat sheets of a radiopaque material (e.g., Pt—Ir) with the slits or cutouts removed from the flat sheet (e.g., by laser cutting, etc.). Increasing the number of cutouts or slits can increase the flexibility of the imaging marker in the axial direction. Therefore, the embodiment of FIG. 8A, with the fewest number of cutouts or slits, is less flexible in the axial direction than the embodiment of FIG. 8B, which is less flexible in the axial direction than the embodiment of FIG. 8C.

The embodiment of FIG. 8D provides another imaging marker with increased flexibility in the axial direction. However, this embodiment is formed in a serpentine pattern so as to provide gaps 26d in the serpentine pattern where the pattern switches back in a direction orthogonal to the axial direction of the imaging marker (and funnel). It should be understood that reducing the thickness of the serpentine pattern and therefore increasing the number of “switchbacks” of the pattern can increase the axial flexibility of the imaging marker, similar to how increasing the number of slits or cutouts in the embodiments of FIGS. 8A-8C increased flexibility in the axial direction. In some embodiments, it should be noted that the serpentine pattern can be arranged such that there are no free ends or sharp corners/edges that may poke or puncture through the compliant material of the funnel during compression/expansion. As shown in FIG. 8D, the ends 28 of the serpentine pattern can be tucked or folded back within the serpentine pattern, such that the imaging marker does not terminate in free ends on either axial end of the marker. Similarly, the embodiments of FIGS. 8A-8C also lack free ends or termination points so as to prevent the imaging markers from poking through or puncturing the funnel during compression and expansion.

In some embodiments, the markers can include orientation features or designs that are asymmetric along the axial or longitudinal axis to provide an indication to the user as to an orientation of the thrombectomy device/funnel, and whether a specific imaging marker is being viewed from above or below the imaging marker. For example, referring to the embodiment of FIG. 8D, the markers can include orientation features that are asymmetric along the axial or longitudinal axis, such as gaps 26d on one side of the marker and straight edges or sections 29 along another side of the marker. Alternatively, the shape of the marker itself can be asymmetric along the axial or longitudinal axis, such as having an “L” shape, a “S” shape, a bent or “U” shape, or any other tortuous, bent, or crooked shape or feature along the longitudinal axis of the marker that makes the marker asymmetric along the longitudinal axis.

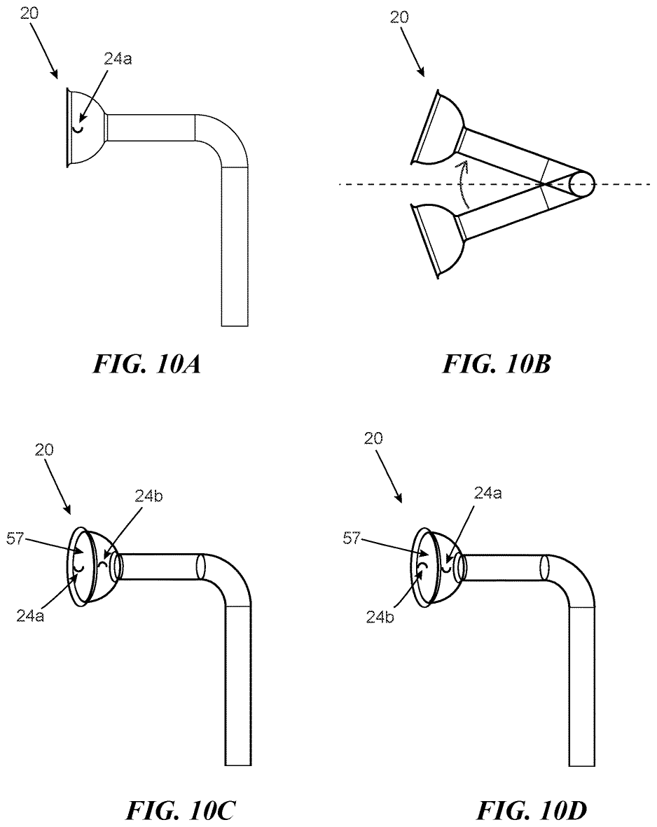

FIG. 10A shows an example of a view of a thrombectomy device having a funnel 20 with one or more markers disposed on or within the funnel. This view of the device as it would be seen in the real-world, such as sitting on a table. Therefore, in this view, only marker 24a is visible to the user, since the marker is positioned on the top surface of the funnel with respect to the user. Additional markers are not visible in this view. For example, another marker positioned on the other side of the funnel (resting against the table) is not visible to the user. The marker in FIG. 10A has a shape that is asymmetric along its longitudinal axis. All markers in this device can share the same shape and same orientation relative to the funnel. For example, if the user were to pick the device up off the table and rotate the funnel about its longitudinal axis, all markers would have the same orientation as that of marker 24a.

FIG. 10B shows a top-down view of the same device, as the device is steered between two positions across an imaging plane 59. FIGS. 10C-10D illustrate fluoro views corresponding to the two positions of the device of FIG. 10B when it is steered across the fluoro imaging plane. For example, FIG. 10C corresponds to the device of FIG. 10B when it is positioned on the side of the imaging plane 59 towards the top of the page, and FIG. 10D corresponds to the device of FIG. 10B when it is positioned on the side of the imaging plane 59 towards the bottom of the page. It can be seen in this example, that markers 24a and 24b are substantially axially displaced along the funnel when steered out of the imaging plane. Here, the user can know that marker 24a is the marker that is positioned on the surface of the funnel that is closest to the fluoro imager, because the asymmetric orientation or shape of the marker matches the shape of the marker on the bench (e.g., the user knows that when the asymmetric design of marker dips downward, it is the marker on surface closest to the imager). The user can also know that the marker 24b is positioned on the funnel surface that is furthest from the fluoro imager (e.g., the user knows that when the asymmetric design of the marker dips upward, it is the marker on the surface furthest from the imager). Since the markers are axially displaced, the user can know that the funnel and/or thrombectomy device is out of the fluoro imaging plane. In FIG. 10C, the user can identify that marker 24a is positioned distally from marker 24b along the longitudinal axis of the device. This tells the user that, in FIG. 10C, the opening 57 of the funnel is facing out of the imaging plane and away from the fluoro imager. Similarly, in FIG. 10D, the user can identify that marker 24a is positioned proximally from marker 24b along the longitudinal axis of the device. This tells the user that the opening 57 of the funnel is facing out of the imaging plane and towards the fluoro imager in FIG. 10D.

With the asymmetrically shaped markers and this knowledge about how they are oriented on the funnel, the user can then steer the catheter/funnel, and can use knowledge of the marker orientation to determine if the catheter/funnel is being steered in or out of the imaging plane and the direction that the funnel is pointing.

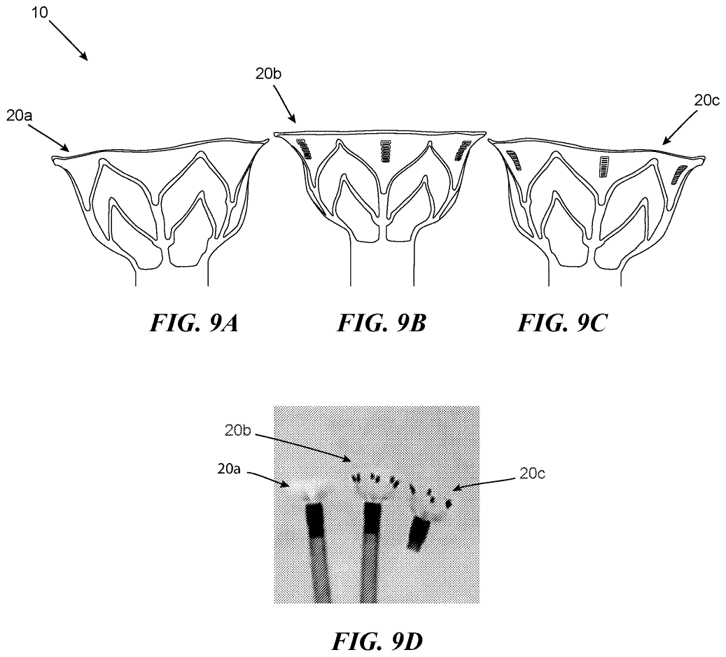

FIGS. 9A-9D illustrate various funnel embodiments with or without the imaging marker embodiments described above. FIG. 9A illustrates a funnel 20a of a thrombus removal device that does not include an imaging marker embedded in or disposed on or near the funnel. FIG. 9B illustrates a funnel 20b of a thrombus removal device that includes a serpentine patterned marker disposed within or embedded within the funnel. This can be, for example, the embodiment described above in FIG. 8D. In this embodiment, one or more imaging marker(s) are placed within the compliant material of the funnel just distal to the funnel frame. In this embodiment, the imaging markers are placed in between the peaks of the chevron pattern of the funnel frame. FIG. 9C illustrates a similar embodiment to that of FIG. 9B, except the imaging marker comprises not a serpentine pattern, but instead a flat sheet of Pt—Ir material, such as one of the imaging markers described above in FIGS. 8A-8C. The imaging marker can include slits or cutouts that can provide additional axial flexibility to the imaging marker.

FIG. 9D shows three different funnel configurations under real-time imaging (e.g., fluoroscopy or ultrasound). Funnel 20a on the left of FIG. 9D shows a funnel that does not include any radio-opaque imaging markers. As shown, a user viewing the real-time imaging cannot ascertain any details about the location of the funnel within the body. In contrast, funnels 20b and 20c of FIG. 9D show funnels with imaging markers embedded in the compliant material of the funnel and supported only by the compliant material. The funnel 20b includes the serpentine-shaped imaging marker of FIG. 8D, and the funnel 20c includes the flat sheet imaging markers of FIGS. 8A-8C (e.g., with laser cut slits).

Any of the embodiments described herein can include imaging markers on other aspects of the device to assist with imaging and placement/delivery. For example, in some embodiments, imaging or radiopaque markers can be placed on the catheter shaft, such as on or near a distal tip of the shaft. As described herein, the device may include an expandable element such as a funnel disposed at or near distal end, with at least one radiopaque marker carried by the expandable element. For example, the at least one radiopaque marker may be embedded within or supported only by a compliant material of an expandable funnel. In this embodiment, a radiopaque gap exists between the radiopaque distal end of the elongate shaft and the radiopaque marker(s) carried by the expandable element. In some examples, the radiopaque gap comprises a portion of the device that is not clinically visible under fluoroscopy imaging.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A medical device, comprising:
an elongate shaft comprising a distal end; and
an expandable funnel disposed at or near the distal end, the funnel comprising a compliant material and at least one imaging marker disposed or encapsulated within the compliant material.

2. The medical device of claim 1, further comprising a funnel frame disposed within the compliant material.

3. The medical device of claim 1, wherein the at least one imaging marker is supported only by the compliant material.

4. The medical device of claim 2, wherein the at least one imaging marker is supported only by the compliant material and not by the funnel frame.

5. The device of claim 1, wherein the at least one imaging marker comprises a radiopaque material.

6. The device of claim 1, wherein the at least one imaging marker comprises platinum iridium.

7. The device of claim 2, wherein the at least one imaging marker comprises a coil, wherein the coil is wound around at least a portion of the funnel frame.

8. The device of claim 1, wherein the funnel frame comprises a shape memory material.

9. The device of claim 1, wherein the funnel frame includes a distal end arranged in a chevron pattern.

10. The device of claim 9, wherein the at least one imaging marker is disposed around at least one segment of the chevron pattern.

11. The device of claim 1, wherein the at least one imaging marker comprises a circumferential coil disposed near a distal end of the funnel.

12. The device of claim 2, wherein the at least one imaging marker comprises a plurality of imaging markers embedded in the compliant material at least partially distal to the funnel frame.

13. The device of claim 1, wherein the at least one imaging marker is arranged generally along a longitudinal axis of the medical device.

14. The device of claim 1, wherein the at least one imaging marker includes one or more axial flexibility features configured to improve or increase an axial flexibility of the at least one imaging marker.

15. The device of claim 14, wherein the one or more axial flexibility features comprises a slit or cutout that is orthogonal to a longitudinal axis of the medical device.

16. The device of claim 1, wherein the one or more imaging markers comprise a serpentine pattern.

17. The device of claim 16, wherein the serpentine pattern includes gaps between the serpentine pattern than are generally orthogonal to a longitudinal axis of the one or more imaging markers.

18. The device of claim 16, wherein the serpentine pattern does not include any free ends.

19. The device of claim 1, wherein the one or more imaging markers are formed from a flat sheet of a Pt—Ir material.

20.-45. (canceled)
